(19) United States
(12) Patent Application Publication
(10) Pub. No.: US 2021/0121245 A1
(43) Pub. Date: Apr. 29, 2021

(54) SURGEON INTERFACES USING AUGMENTED REALITY

(71) Applicant: TransEnterix Surgical, Inc., Morrisville, NC (US)

(72) Inventors: Kevin Andrew Hufford, Cary, NC (US); Matthew Robert Penny, Holly Springs, NC (US); Sevan Abashian, Morrisville, NC (US); Bryan Peters, Morrisville, NC (US)

(21) Appl. No.: 17/064,334

(22) Filed: Oct. 6, 2020

(51) Int. Cl. A61B 34/00; A61B 34/37; G02B 27/01

(52) U.S. Cl. CPC A61B 34/25; A61B 34/74; A61B 2034/252; G02B 27/017; A61B 34/37

(57) ABSTRACT

A system for visualizing and controlling tasks during surgical procedures, the system comprising a display for displaying information to a user and a user interface operable to generate input commands to a surgical system in response to surgeon movement.

### BACKGROUND

There are various types of surgical robotic systems on the market or under development. Some surgical robotic system use a plurality of robotic arms. Each arm carries a surgical instrument, or the camera used to capture images from within the body for display on a monitor. Other surgical robotic systems use a single arm that carries a plurality of instruments and a camera that extend into the body via a single incision. These types of robotic system use motors to position and orient the camera and instruments and, where applicable, to actuate the instruments. Input to the system is generated based on input from a surgeon positioned at master console, typically using input devices such as input handles and a foot pedal. Motion and actuation of the surgical instruments and the camera is controlled based on the user input. The image captured by the camera is shown on a display at the surgeon console. Examples of surgical robotics systems are described in, for example, described in WO2007/088208, WO2008/049898, WO2007/088206, US 2013/0030571, and WO2016/057989, each of which is incorporated herein by reference.

The Senhance Surgical System from TransEnterix, Inc. includes, as an additional input device, an eye tracking system. The eye tracking system detects the direction of the surgeon's gaze and enters commands to the surgical system based on the detected direction of the gaze. The eye tracker may be mounted to the console or incorporated into glasses (e.g. 3D glasses worn by the surgeon to facilitate viewing of a 3D image captured by the camera and shown on the display). Input from the eye tracking system can be used for a variety of purposes, such as controlling the movement of the camera that is mounted to one of the robotic arms.

The present application describes various surgeon interfaces incorporating augmented reality that may be used by a surgeon to give input to a surgical robotic system.

### BRIEF DESCRIPTION OF THE DRAWINGS

FIGS. 1 and 2 schematically depict types of information that may be integrated into a display provided for use with a robotic surgical system.

FIG. 3 shows an embodiment of an augmented reality system for use with a surgical system.

FIG. 4 shows an embodiment of a user input device for use with a surgical system.

FIG. 5A shows an embodiment FIG. 5A using a projector mounted above the patient table;

FIG. 5B shows a display system for use with a surgical system.

FIGS. 6 through 8 show three embodiments of visualization and user interface configurations for use with a surgical system.

### DETAILED DESCRIPTION

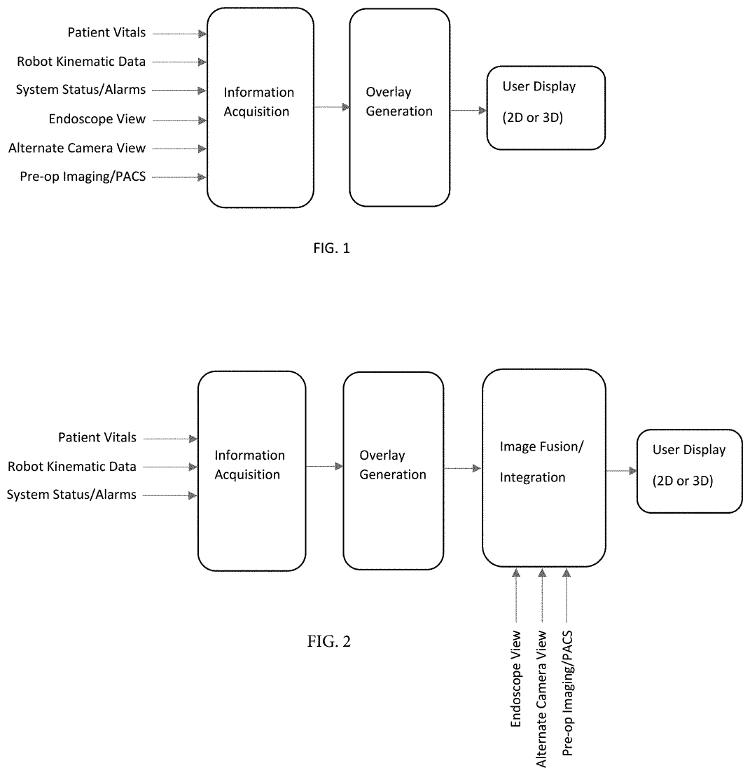

This application describes systems that enhance the experience of surgeons and/or surgical staff members by providing an enhanced display incorporating information useful to the surgeon and/or staff. FIGS. 1 and 2 give an overview of the types of information that may be integrated into a display provided for use with a robotic surgical system, but the types of information and imaging sources that may be integrated are not limited to those specified below.

In the FIG. 1 diagram, an overlay is generated from a variety of information sources and imaging sources. For a transparent augmented reality display, the generated image may not necessarily fill the entire screen nor be completely opaque. In the FIG. 2 diagram, an overlay is placed over at least one imaging source. This approach would be necessary for a virtual reality-type display, in which the back of the display is opaque.

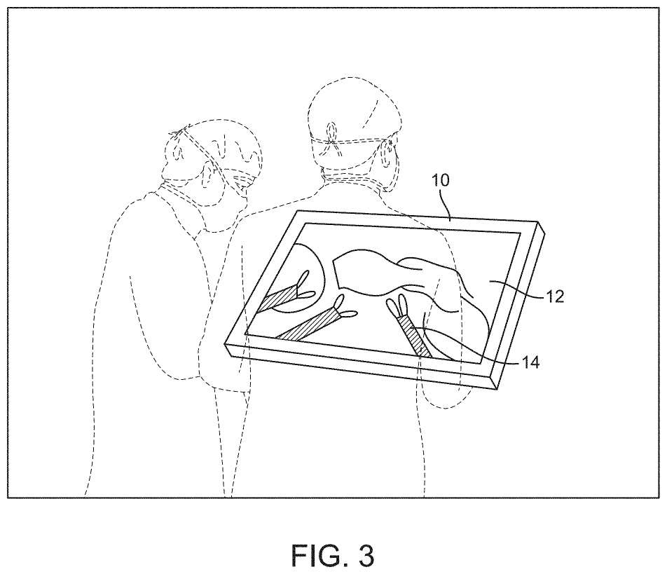

Referring to FIG. 3, in a first embodiment, a semi-transparent screen or monitor 10 is mounted above the operating room table that is used to support a patient. The monitor 10 may be movable away from the table to facilitate loading of the patient onto the table. The monitors height can be manipulated such that operating staff can work on the patient's abdomen unimpeded. Additionally, when desired, the monitor can be lowered above the patient such that the surgeon's hands (and those of other operating room staff) can work beneath the monitor and the image displayed on the monitor is visible to all of the patient-side operating room staff.

The monitor 10 positioned above the patient can be used for displaying various information about the procedure and the patient. For example, the monitor can be used to display real-time 3D scanned images, video from operative cameras (laparoscopic, endoscopic, etc), patient vital signs, procedural information (steps, instruments being used, supply counts, etc).

Beneath or on the underside of the monitor 10 (i.e. the face oriented towards the patient) is a user interface 12 for giving input to the surgical system to control the surgical instruments that are operated by the system. When surgeon places his/her hands under the screen, he can manipulate the system through the user interface. This interface could require the surgeon to grasp and manipulate a handle-type device that functions as a user-input device, or the surgeon interface could comprise a series of cameras positioned to capture his/her hand gestures. In the latter example, user hand movements beneath the monitor are tracked by the camera system (e.g. using optical tracking). The detected movements are used as input to cause the system to direct corresponding movement and actuation of the robotic surgical instruments within the body. In this way, the surgeons can view the operative site and simultaneously manipulate instruments inside the body. Graphical depictions or images 14 of the surgical instruments (or just their end effectors) are shown on the display.

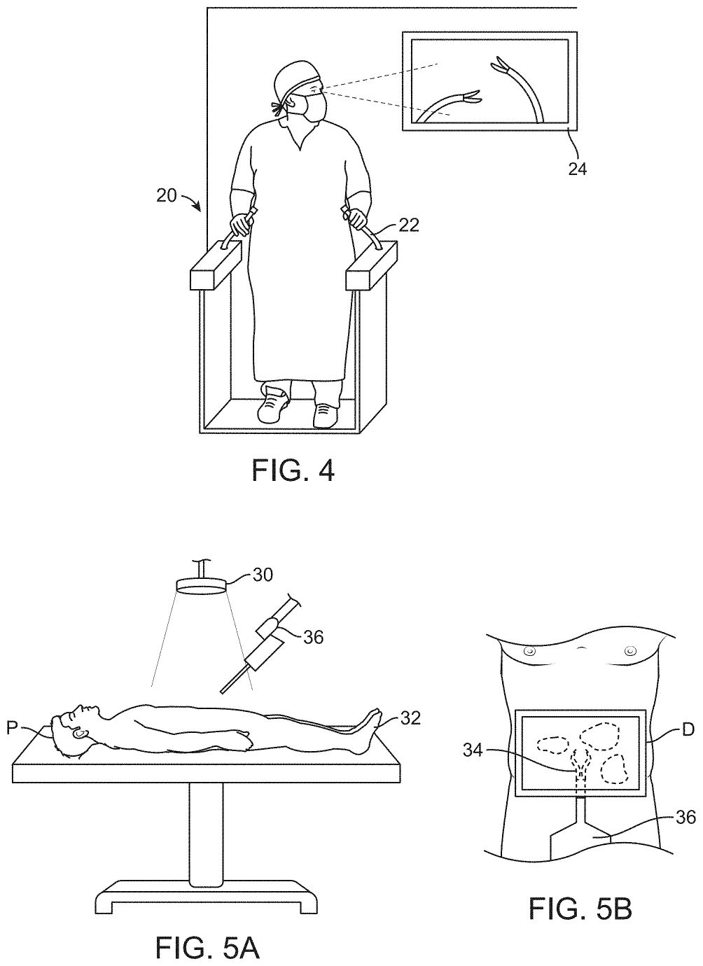

Referring to FIG. 4, a second embodiment includes a user interface 20 that mimics flexible robotic instruments of a type that may be disposed within the patient. The system is designed to allow the surgeon to stand between two controllers 22, each of which has degrees of freedom similar to those of the flexible instruments. The user manipulates the controllers to command the system to direct motion of the instruments, while observing the procedure on the camera display 24. The similarities in the nature of the motion of the controllers and that of the instruments helps the surgeon to correlate the desired movement of the instruments to the necessary movement of the controllers to achieve that instrument motion.

A third embodiment, shown in FIG. 5A, makes use of a projector 30 mounted above the patient table 32. The projector projects the image captured by the camera/surgical landscape onto the drape D covering patient P as shown in FIG. 5B, or onto a screen (not shown) above the patient. The projected image is aligned with and shows projected images 34 of instruments in the positions within the body, and other anatomical landmarks that are within the patient's body. This allows the surgeon and staff to look down at the patient and get an anatomical sense of where they are working. These drawings show the system used with a single port type of robotic system, in which an arm 36 supports multiple surgical instruments, however it may be used with other types of surgical systems as well.

A fourth embodiment utilizes a variation of “smart glasses” that have an inset screen, such as the Google Glass product (or others available from Tobii, ODG, etc). The display on the glasses may be used to display patient vitals, procedure steps, views captured by auxiliary (or primary) cameras/scopes, or indicators of locations of instruments within the body.

The specific embodiment of these glasses incorporates both externally facing cameras as well as internally facing cameras. See, for example, US Patent Application 2015/0061996, entitled “Portable Eye Tracking Device” owned by Tobii Technology AB and incorporated herein by reference. The externally facing cameras would be used to track surgeon gaze or movement around the operating room (i.e. is s/he looking at an arm, or a monitor, or a bed, etc). The internally facing cameras would track the surgeon's eye movement relative to the lenses themselves. The detected eye movement could be used to control a heads-up-display (HUD) or tracked as a means to control external devices within the view of the externally facing camera. As an example, these glasses could also be used to direct movement of the laparoscopic camera for panning or zoom by measuring the position and orientation of the wearer relative to an origin in the operating room space, or through measuring the position of the pupils relative to an external monitor or the HUD within the glasses themselves. As another example, input from the external camera would be used to detect what component within the operating room the user was looking at (a particular arm, as identified by shape or some indicia affixed to the arm and recognized by the system from the sensed external camera image), causing the system to call up a menu of options relative to that component on the HUD of the glasses themselves, finally allowing the user to select a function for that component from that menu of options by focusing her gaze on the desired mention option.

In a variation of the fourth embodiment, user input handles of the surgeon console might be replaced with a system in which the user's hands or “dummy” instruments held by the user's hands are tracked by the externally facing camera on the smart glasses. In this case, the surgeon console is entirely mobile, and the surgeon can move anywhere within the operating room while still commanding the surgical robotic system. In this variation, the externally facing camera on the glasses is configured to track the position/orientation of the input devices and the system is configured to use those positions and orientations to generate commands for movement/actuation of the instruments.

A fifth embodiment comprises virtual reality (“VR”) goggles, like those sold under the name Oculus Rift. These goggles differ from those of the fourth embodiment in that they are opaque and the lenses do not permit visualization of devices beyond the screen/lens. These goggles may be used to create an immersive experience with the camera/scope's 3D image output as well as the user interface of a surgical system.

In one variation of this embodiment, the VR goggles are worn on the operator's head. While wearing VR goggles, the surgeon's head movements could control the position/orientation of the scope. In some embodiments, the goggles could be configured to display stitched-together images from multiple scopes/cameras.

In a second variation of this embodiment, the VR goggles might instead be mounted at eye level on the surgeon console, and the surgeon could put his/her head into the cradle of the goggles to see the scope image in 3D for an immersive experience. In this example, the surgeon console might also have a 2D image display for reference by the user at times when the 3D immersive experience is not needed. In some implementations of this embodiment, the goggle headset is detachable from the surgeon console, permitting it to be worn as in the first variation (described above).

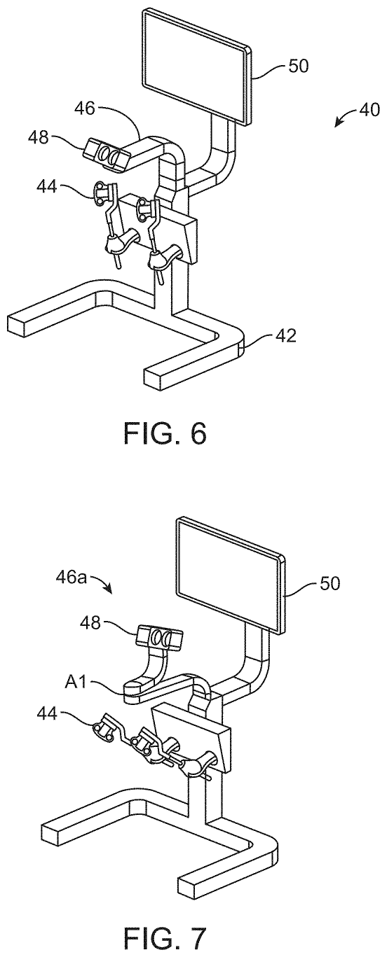

FIG. 6 shows a surgeon console 40 which comprises a base 42, control input devices 44, a mounting arm 46, and a 3D display, which may be VR goggles 48. In some variations of the implementation, an auxiliary monitor 50 is also attached to the console. In this implementation, the mounting arm is rigid. The control input devices 44 are grasped and manipulated by the surgeon to generate input that causes the robotic system to control motion and operation of surgical instruments of the robotic system.

FIG. 7 shows a surgeon console 40a, which uses a mounting arm 46a having a single rotary axis A1 nominally aligned with the user's neck to for natural side-to-side motion, referred to as yaw. This allows the user to move his/her head to give input that will cause the endoscope to move from side-to-side within the patient's body, or otherwise alter the display to change the user's viewpoint of the operative site, such as by moving within a stitched image field, or to switch between various imaging modes. In other variations (not shown), this single axis may be aligned with the natural tilt motion of the head, referred to as pitch.

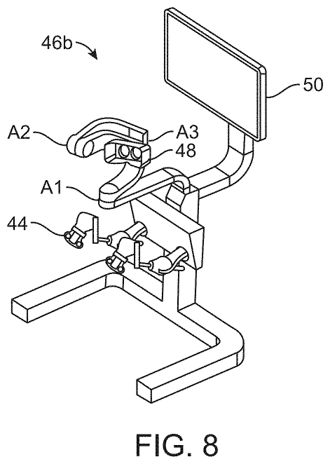

FIG. 8 shows a console for which the mounting arm 46b for the 3D display allows the user to rotate about more than one axis. In particular, the yaw axis A1 is at least nominally centered about the neutral axis of the user's neck, and the pitch axis is also positioned to accommodate natural head tilt. A roll axis A2 is centered about the center of the visual field, which may be used to roll the camera and adjust the horizon. Two-axis versions of this implementation may eliminate the roll axis. In some implementations that incorporate the roll axis, this roll axis may just be passive for ergonomic comfort of the user as the head moves side to side.

In some implementations, fore/aft head motion of the user's head may also be allowed via a prismatic joint in-line with the 3D display and roll axis and may be used as input to the system to control zooming of the endoscope.

The mounting arm implementations shown in FIGS. 7-8 use serial, rotary linkages to provide the desired degrees of freedom, but are not limited to these types of linkages. Other means of providing the desired degrees of freedom may include, but are not limited to, four-bar linkage mechanisms, prismatic joints, parallel kinematic structures, and flexural structural elements.

As noted, in FIGS. 7 and 8, the position and orientation of the headset 48 relative to an origin can be tracked, either through external cameras looking at the headset, or through cameras built into the headset, through an inertial measurement unit (IMU) positioned on the headset itself, or through encoders or other sensors incorporated into the axes of the mounting structure. Instead of a full-featured IMU, accelerometer(s), gyroscope(s), or any combination may also be used.

The movement of the headset could be used as an input to the system for repositioning of instruments or a laparoscope, as an example. The movement of the headset may be used to otherwise alter the user's viewpoint, such as moving within a stitched image field, or to switch between various imaging modes.

1-34. (canceled)

35. A user interface for a surgical system that robotically manipulates at least one surgical device, the user interface comprising:
a monitor configured to display real-time video images of an operative site information and to display surgical procedure information as an overlay on the displayed video images;
a user input disposed on an underside of the monitor, the user input operable to generate input signals to the surgical system in response to surgeon movement, the surgical system responsive to the input systems to control movement of the surgical device.

36. The user interface of claim 35, where the monitor and user input are positionable suspended above an operating room table.

37. The user interface of claim 35, wherein the displayed surgical procedure information comprises surgical steps.

38. The user interface of claim 35, wherein the displayed surgical procedure information comprises information concerning instruments in use in a surgical procedure.

39. The user interface of claim 35, wherein the displayed surgical procedure information comprises patient vital signs.

40. The user interface of claim 35, where the user interface is manually manipulatable by the operator to generate user input to the system.

41. The user interface of claim 35, where the user input includes at least one image sensor positioned to detect movements of an operator's hand or an object held by the operator's hand behind the monitor.

42. A user interface for a surgical system that robotically manipulates at least one surgical device, the user interface comprising:
a head mounted display configured to display real-time video images captured by at least one camera at an operative site;
a surgeon console including a user input device operable to generate input signals to the surgical system in response to surgeon movement, the surgical system responsive to the input systems to control movement of the surgical device.

43. The user interface of claim 42, wherein the surgeon console further includes a monitor, the head mounted display selectively configured to display the real-time video images.

44. The user interface of claim 43, wherein the surgeon console includes a support and wherein the head mounted display is mounted to the support.

45. The user interface of claim 44, wherein the head mounted display is removably mounted to the support for mobile use by the operator.

46. The user interface of claim 42, where the system is configured to track motion of the head mounted display to receive said motion as user input to the surgical system.

47. The user interface of claim 46, wherein the system is configured to direct a change of a view of the at least one camera.

48. The user interface of claim 46, wherein the system is configured to direct a change of an imaging mode of the at least one camera.

49. The user interface of claim 46, wherein the system is configured to direct movement within a stitched image view.
